(19) United States
(12) Patent Application Publication
(10) Pub. No.: US 2008/0255654 A1
(43) Pub. Date: Oct. 16, 2008

(54) SYSTEM FOR DELIVERING A STENT

(75) Inventors: Stephen Hebert, San Francisco, CA (US); Marc-Alan Levine, Pottstown, PA (US)

Correspondence Address: BERENBAUM, WEINSHIENK & EASON, P.C, 370 17TH STREET, SUITE 4800, DENVER, CO, 80202, US

(73) Assignee: BAY STREET MEDICAL, San Francisco, CA (US)

(21) Appl. No.: 12/053,099

(22) Filed: Mar. 21, 2008

Related U.S. Application Data
(60) Provisional application No. 60/919,371, filed on Mar. 22, 2007.

(51) Int. Cl. A61F 2/84; A61F 2/82

(52) U.S. Cl. 623/1.11; 623/1.34

(57) ABSTRACT

A stent delivery system including an elongate member having a proximal segment and a distal segment, the distal segment having a first coil section, a second coil section, an intermediate coil section located between the first and second coil sections and a plastic material overlying one or more of the coil sections.

### CROSS-REFERENCE TO RELATED APPLICATION

This application claims priority as a Non-Provisional of U.S. Provisional Patent Application No. 60/919,371 for “System for Delivering a Stent” of Stephen Hebert et al, filed Mar. 22, 2007, hereby incorporated by this reference in its entirety as though fully set forth herein.

### BACKGROUND

1. Technical Field

This application relates to a system for delivering a stent, and more particularly to a delivery guide having one or more polymeric coated distal segments. The application also relates to a delivery system wherein a stent is mounted on a reduced diameter area of the delivery wire to reduce the overall profile of the system.

2. Background of Related Art

Intravascular stents are used for treatment of vascular stenosis. One type of stent is a balloon expandable stent which is mounted over a balloon. Inflation of the balloon expands the stent within the vessel to dilate the stenosis. Plastic deformation of the stent retains the stent in its expanded configuration. Another type of stent is of the self-expanding type which is composed of a shape memory material. Self-expanding stents are typically compressed within a sheath and when exposed from the sheath automatically moves toward an expanded shape memorized position within the vessel.

These stents are generally delivered to the area of stenosis or an aneurysm by a catheter which is inserted over a guidewire. For balloon expandable stents, the balloon is mounted at the distal end of the catheter and is expanded by injection of fluid through a lumen of the catheter. Expansion of the balloon expands the overlying stent. For self-expanding stents, these stents are typically compressed against the outer surface of a catheter and subsequently placed inside a sheath for delivery to a treatment site.

The applicants in an earlier application recognized that utilizing a catheter with a stent mounted thereon did not enable access to small vessels. To reduce the cross-sectional dimension of the stent delivery system, the applicants developed a system for placing a stent on a delivery wire (e.g., guidewire) or hypotube, rather than on or within a catheter (which was inserted over a guidewire), thereby eliminating the larger dimensioned catheter. This system is described in commonly assigned U.S. Pat. No. 6,989,024, the entire contents of which are incorporated herein by reference, which discloses a stent mounted on a guidewire or hypotube. The stent is mounted on a reduced diameter portion, resulting in an overall reduced profile. Proximal and distal radiopaque marker bands, functioning as proximal and distal stops for the stent, are also described for certain embodiments. Reduced profile delivery systems are also disclosed in commonly assigned co-pending application Ser. Nos. 11/703,341 and 11/703,342, both filed on Feb. 7, 2007. The entire contents of these applications are incorporated herein by reference.

The apparatus and method disclosed in the '024 patent is effective in accessing smaller vessels and delivering a stent to such vessels. The present application provides improvements and variations to the stent delivery systems disclosed in the '024 patent.

### SUMMARY OF THE INVENTION

The present invention in one aspect provides a stent delivery system comprising a coil of varying diameter covered by a plastic material. The varying diameter results in the plastic cover, optionally of varying diameter, to create a reduced diameter region for receiving a stent to reduce the overall profile of the delivery system. Proximal and distal stops formed by walls of the plastic cover could be provided to limit axial movement of the stent mounted thereon.

In one embodiment, the reduced diameter region is a tapered region. In another embodiment, it is a stepped region which can be formed, for example, by a smaller diameter coiled region stepping down from a larger first coil portion then stepping up to a larger second coil portion. Gaps can optionally be formed between coil portions to enhance imaging.

The stent is positioned on the reduced diameter portion of the guidewire and is preferably covered during insertion to the site. Uncovering the stent enables it to move to an expanded configuration. The stent can take a variety of forms and be of various materials, expanding to engage the walls of the vessel and allow blood flow therethrough.

### BRIEF DESCRIPTION OF THE DRAWINGS

Preferred embodiments of the present disclosure are described herein with reference to the drawings wherein:

FIG. 1A is a side view of an embodiment of the stent delivery guide of the present invention having a coil of varying diameter;

FIG. 1B is a side view of another embodiment of the stent delivery guide of the present invention having a coil of varying diameter;

FIG. 2A is a side view of another embodiment of the stent delivery guide of the present invention having gaps between the coils;

FIG. 2B is a schematic view showing the imaging of the stent on the guide of FIG. 2A;

FIG. 3 is a side view showing another embodiment of the stent delivery guide of the present invention having a coil of uniform diameter and showing a stent mounted thereon;

FIG. 4 is a side view of another embodiment of the stent delivery guide of the present invention having molded indentations to enhance stent retention;

FIG. 5 is a side view of another embodiment of the present invention, similar to FIG. 3 except having radiopaque marker bands; and

FIG. 6 is a side view showing another embodiment of the stent delivery guide of the present invention having a plastic material proximal to the coil section.

### DETAILED DESCRIPTION OF PREFERRED EMBODIMENTS

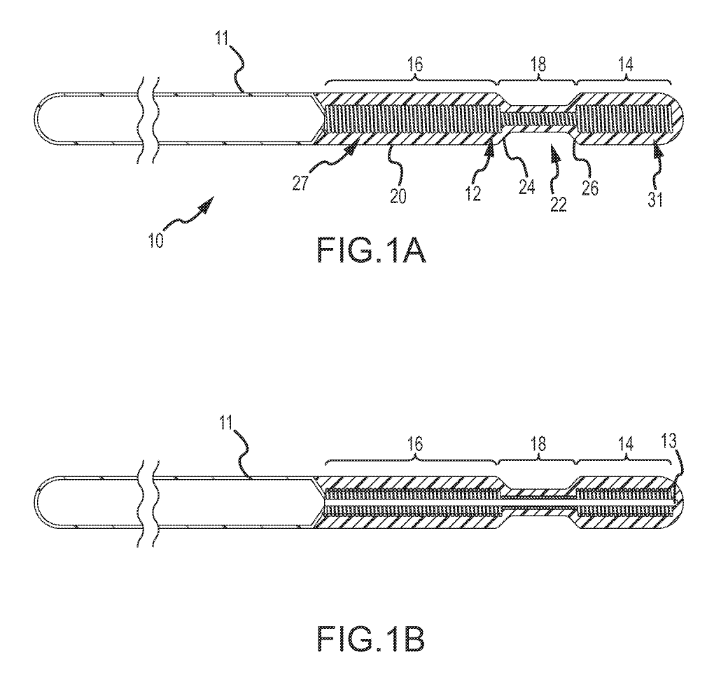

Referring now in detail to the drawings wherein like reference numerals identify similar or like components throughout the several views, a first embodiment of the stent delivery system of the present invention is shown in FIG. 1A. In this embodiment, stent delivery system is represented generally by reference numeral 10 and includes a guidewire or guide comprising a coil 12 covered by a plastic cover 20 that extends distally from an elongate proximal segment 11 of guide 10. Proximal segment 11 may comprise any of a variety of materials or combinations thereof, such as, for example, metal, plastic, composites, etc. As shown, the coil 12 has a distal region 14 of a first diameter and a proximal region 16 of a second diameter, which is preferably substantially equal to the first diameter, although in alternate embodiments it could be of larger or smaller diameter. The middle or intermediate coil region 18 has a smaller diameter than the proximal and distal regions 16, 14 to create a reduced diameter region for mounting of the stent (not shown). The plastic cover 20 can be in the form of heat shrink tubing attached to the distal end of the core and to the coils. Materials of the plastic cover can include for example Teflon, PTFE, FEP, EPTFE, Paralene, Polyofin, Nylon and Pebax. Also, instead of a shrink wrap, a plastic coating such as Polyurethane, Pebax, Nylon, Polyimide, PVC, Escorolene, HDPE and LDPE could be provided. The shrink wrap maintains a reduced diameter area 22 over the reduced diameter intermediate coil region 18. Therefore, when a stent is mounted on the guide 10 over area 22, a reduced profile system is provided.

A proximal stop is formed by edge 24 where the plastic cover 20 transitions from its larger diameter region 27 to its reduced diameter region 22. Similarly, a distal stop is formed by edge 26 where the plastic cover 20 transitions from its reduced diameter region 22 to its larger diameter region 31. Preferably, the angle transitions as shown. Distal and proximal stops 26, 24 limit axial (distal and proximal) movement of the stent as the stops have a transverse cross-section or outer diameter larger than the reduced diameter portion 22 of guidewire 10. By mounting the stent on the guidewire, and on the reduced diameter region, an overall reduced profile of the delivery system is achieved. The advantages of such reduced profile mounting in this embodiment as well as the other embodiments disclosed herein are described in detail in commonly assigned U.S. Pat. No. 6,989,024 and co-pending U.S. application Ser. No. 11/248,362, filed Oct. 11, 2005, the entire contents of which are incorporated herein by reference.

Although shown as a step down to a reduced diameter followed by a step up to a larger diameter, alternatively, to provide a reduced diameter stent mounting region, the coil could be tapered in the region underlying the stent and/or the plastic coating can be applied to provide a tapered region of reduced diameter to receive the stent. The plastic coating may also have a tapered profile when applied to the other coil regions and may be applied in way to provide a smooth transition in flexibility along the coil regions.

Note the stents of the embodiments disclosed herein can be composed of shape memory, stainless steel or other metals or metal composites and of radiopaque material. In other embodiments one or more surface characteristics of the plastic cover (e.g., tackiness) are modified to enhance stent retention.

An advantage of the present invention is that it provides greater flexibility in the design of the distal segment of guide 10. That is, the flexibility along the length of the distal segment can be easily tailored to meet specific device requirements. For example, the flexibility, pushability or steerability of the distal segment regions can be manipulated by altering one or more of the following parameters: coil pitch, coil material, plastic cover thicknesses, plastic cover material, temperature and/or chemical treatment of one or both coil and plastic cover materials, doping of the plastic cover, providing cuts and/or notches within the plastic cover, etc. This is important since the type and length of stents mounted on delivery systems of this type will vary depending on the type and length of the lesion being treated and, as a result, will affect the flexibility, pushability and steerability of the distal segment of the system. As noted above, doping of one or more portions of the plastic cover can be used to locally alter the physical properties of the plastic. Doping may also include integrating within the plastic radiopaque materials that enhance the visibility of the device under fluoroscopy.

In an alternative embodiment, as shown in FIG. 1B, the distal segment of guide 10 includes a core member 13 that extends distally from the proximal segment 11 and through one or more of coil regions 16, 18 and 14. Core member 13 may be attached to or integral to proximal segment 11. The core member may have a uniform cross-section or may be have varying cross-sections in the form of one or more stepped or tapered portions. Manipulation of the core member properties, e.g., material, thickness, temperature and/or chemical treatment, the inclusion of cuts or notches in the external surface, etc., can be used to obtain desired device performance characteristics (e.g., flexibility, pushability, steerability, etc.).

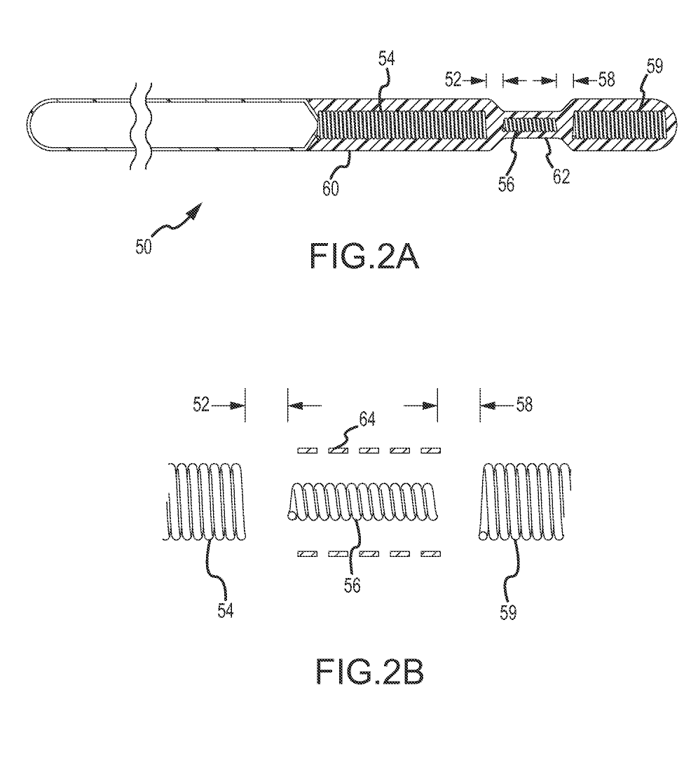

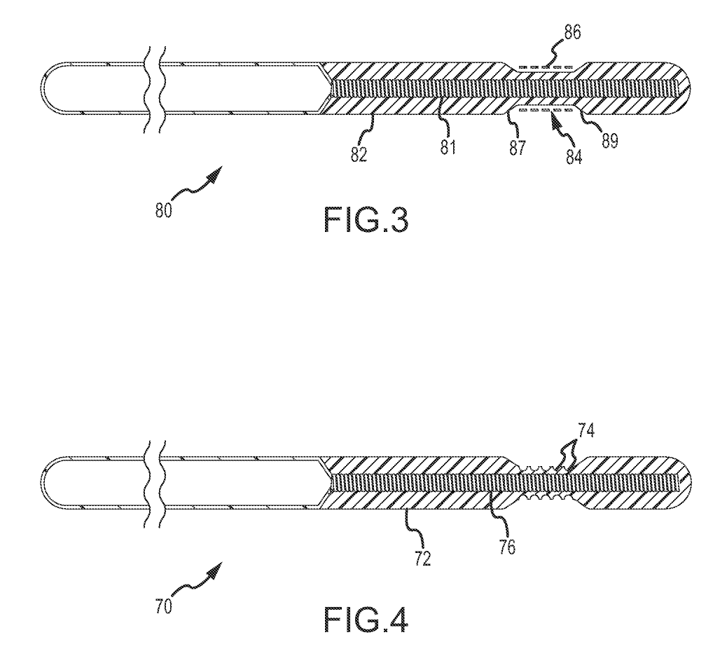

FIG. 2A illustrates an alternate embodiment of the stent delivery system wherein gaps are provided between the three coil sections. More specifically, a gap 52 is provided between proximal coil section 54 and intermediate coil section 56 and a gap 58 is provided between coil section 56 and distal coil section 59. The intermediate coil section, as in the embodiments of FIGS. 1A and 1B, can have a smaller diameter than the proximal and distal coil sections 54, 59, with the coil sections 54 and 59 preferably of substantially equal diameter, although alternatively they could be of different diameters. Alternatively, the coil sections can be of substantially uniform diameter as shown with the plastic cover melted as described below with respect to the embodiment of FIG. 3. The gaps 52, 58 are preferably created by stretching the coil during manufacture. The gaps enhance imaging as shown schematically in FIG. 2B, illustrating the features visualized under fluoroscopy, better highlighting the stent 64. The gaps may also be used to receive radiopaque markers extending longitudinally from one or both ends of mounted stent. Alternatively, the gaps may be configured to receive radiopaque markers positioned on the end cell structures of a mounted stent.

It is also contemplated that the gaps could be created by three separately spaced apart discrete coils retained within the plastic cover. In such embodiments, the middle coil, corresponding to the region where the stent is mounted, could be composed of a different material with a different radiopacity, e.g. enhanced radiopacity to improve imaging in the region of the stent. It is also contemplated that the separate coils could be made of different materials and/or each coil section composed of separate coils. For example, the proximal coil could have one coil, e.g. the distal coil, made of platinum and another coil made of stainless steel. The two coils could be welded or otherwise attached. In one embodiment the platinum could extend about 5 mm adjacent the stent receiving portion of the guidewire and the stainless steel extend about 13 cm, although other dimensions are contemplated.

In the embodiment of FIG. 3, the coil 81 of guidewire 80 is shown having a substantially uniform diameter with the plastic cover 82 placed over the coil. The plastic cover is formed with a reduced diameter region 84 to receive the stent 86. In this embodiment, the plastic cover can be applied by dipping, over extrusion, spraying, or other processes, and then optionally melted in a secondary heat process to form the reduced diameter region 84. The edges 87, 89, proximal and distal to the reduced diameter region 84, create stops to limit axial movement of the stent. It should be appreciated that alternatively the coil can have a reduced region as in the embodiments of FIGS. 1A and 1B.

Another process for forming the reduced region in the plastic cover comprises sliding plastic such as Pebax, Nylon, LDPE, (or other materials) over the coil/core and then sliding heat shrink tubing over the assembly and melting. Once melted, the heat shrink is removed and the reduced region is formed by a die or another heat process.

In the alternate embodiment of FIG. 4, the plastic cover 72 of guidewire 70 has indentations or grooves 74 molded in the outer surface to improve stent retention. The coil 96 can be of any of the foregoing embodiments, e.g. with gaps, of varying diameter, etc.

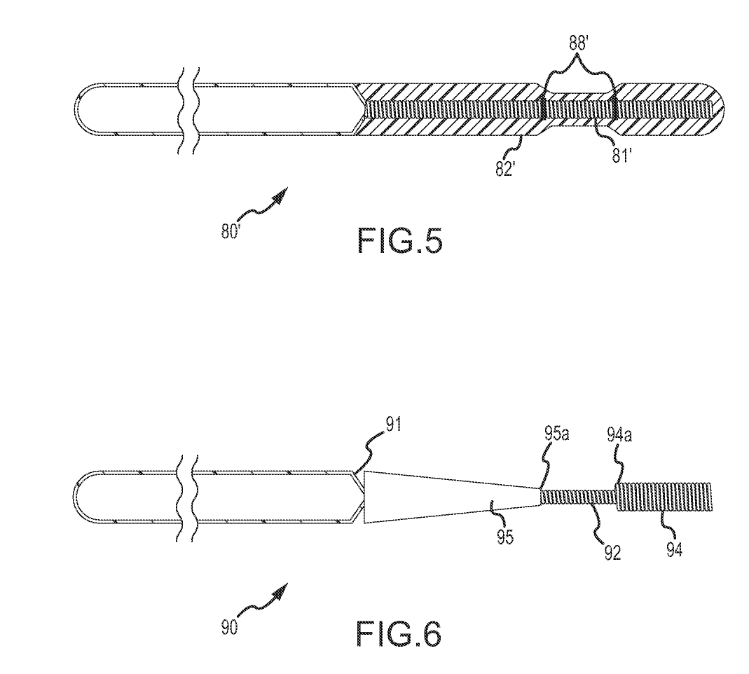

One or more radiopaque markers, such as a marker band 88′, can optionally be provided on the coil 81′ inside the plastic coating 82′ as shown for example in FIG. 5 to enhance imaging. Such marker bands can be used with other embodiments described herein.

In the alternate embodiment of FIG. 6, the guidewire 90 has a reduced diameter coil region 92 to receive a stent (not shown) thereon, a distal larger diameter coil region 94, and a plastic cover 95 proximal of coil region 92 and overlying a distal portion of core 91. Alternatively the coil could extend further proximally so the plastic cover could overlie the coil. The distal edge 95a of the plastic cover 95 forms a proximal stop and the surface 94a of coil region 94 forms a distal stop for the stent. In other embodiments the plastic cover 95 is extended to cover coil region 92.

The delivery guide/wire in any of the foregoing embodiments can have cutouts, as previously described, to increase the flexibility and steerability.

The stent as discussed herein is mounted on the tapered or reduced diameter region of the guide of the various embodiments. A tube, catheter, or sheath (not shown) would be positioned over the guide and the stent to maintain the stent in the compressed position. Relative movement, e.g. retraction of the tube, catheter, or sheath, advancement of the guide, or movement of both in opposite directions, exposes the stent for self-expansion.

The tube, catheter or sheath utilized can have slits for flexibility. They can be composed of a composite material, and can contain a Teflon liner with a soft outer jacket and radiopaque markers to delineate the stent region as well as the end of the devices.

Note, the coil in the foregoing embodiments can be of different lengths and extend, for example, further proximally than shown. Also, the coils or coil section in each of the embodiments can be of substantially uniform diameter or of varying diameter. Further, the sections can be integrally formed by a single coil or formed from two or more coils.

By way of example, the guidewire can preferably have a diameter of about 0.003 inches to about 0.040 inches, and more preferably about 0.016 inches, with the stepped down or reduced diameter area preferably of about 0.0095 inches.

A hydrophilic lubricious coating or PTFE coating could be provided over the guidewire, and if coating is utilized, optionally selected areas of the guidewire could be left uncoated such as the area over which the stent is placed to increase frictional contact.

As discussed above, the delivery systems of the present invention can be inserted into a lumen of an already placed microcatheter or tube with or without a sheath or alternatively can be inserted into the microcatheter or tube (with or without a sheath) before its placement at the surgical/treatment site. In other embodiments, a guide including a sheath which constrains the stent can be delivered to the treatment site without the use of a delivery catheter. Alternatively, the sheath can be placed in the body, and the stent mounted guidewire delivered through the already placed sheath.

The guides described herein may be used as guidewires in applications without a mounted stent with the reduced diameter region adjacent the tip increasing the deflection/flexibility of the tip. Thus, the reduced diameter can provide a guidewire of varying stiffness, e.g. a less stiff distal end.

While the above description contains many specifics, those specifics should not be construed as limitations on the scope of the disclosure, but merely as exemplifications of preferred embodiments thereof. For example, a distal and proximal stop, either integral or attached, and made of a radiopaque material for imaging, could be provided. Further, to provide a reduced diameter mounting region, as an alternative to a stepped region, a taper or cut out region could be provided. The tip of the wire could be shapeable. The plastic cover can extend over a portion of the core a further length than shown and can optionally extend over the entire length of the core. In such embodiments, the proximal segment 11 of the core can be tapered with a plastic cover disposed over the proximal segment having a reverse taper that creates a uniform cross-sectional area along a length, or the entire length of the proximal segment. Alternatively, the tapering of the proximal segment and of the plastic cover may result in a tapered proximal segment or a proximal segment having varying diameters or cross-sections. The coils can also extend different lengths. Those skilled in the art will envision many other possible variations that are within the scope and spirit of the disclosure.

What is claimed is:

1. A stent delivery system including:
an elongate member having a proximal segment and a distal segment, the distal segment having a first coil section, a second coil section, an intermediate coil section located between the first and second coil sections and;
a plastic material overlying one or more of the coil sections.

2. A stent delivery system according to claim 1, wherein the plastic material is a heat shrink tubing.

3. A stent delivery system according to claim 1, wherein the first coil section is a proximal coil section proximal to the proximal segment.

4. A stent delivery system according to claim 3, wherein the intermediate coil section has a diameter less than one or the other or both the diameters of the proximal and distal coil sections.

5. A stent delivery system according to claim 1, wherein the coil sections are of substantially uniform diameter.

6. A stent delivery system according to claim 1, wherein a first gap is formed between the first and intermediate coil section.

7. A stent delivery system according to claim 1, wherein a second gap is formed between the intermediate and second coil sections.

8. A stent delivery system according to claim 6, wherein a second gap is formed between the intermediate and second coil sections.

9. A stent delivery system according to claim 1, wherein the coil sections are composed of a single coil.

10. A stent delivery system according to claim 1, wherein the coil sections are composed of discrete coils.

11. A stent delivery system according to claim 1, wherein the plastic material has a reduced diameter region overlying at least a portion of the intermediate coil section.

12. A stent delivery system according to claim 11, wherein the reduced diameter is formed by a melting process.

13. A stent delivery system according to claim 11, wherein the reduced diameter region includes at least one of a taper and a step down region.

14. A stent delivery system according to claim 1 comprising a core member positioned within one or more of the coil sections.

15. A stent delivery system according to claim 14, wherein the core member comprises one or more tapers.

16. A stent delivery system according to claim 14, wherein the core member comprises one or more steps.

17. A stent delivery system according to claim 1, wherein the plastic material comprises one or more tapers.

18. A stent delivery system according to claim 1, wherein the plastic material comprises one or more steps.

19. A stent delivery system according to claim 1, wherein the plastic material comprises a doped plastic.

20. A stent delivery system according to claim 7, wherein the first gap is configured to receive a radiopaque marker positioned on a stent to be mounted on the stent delivery system.
